(19) United States
(12) Patent Application Publication
(10) Pub. No.: US 2013/0202628 A1
(43) Pub. Date: Aug. 8, 2013

(54) PRODUCTION AND THERAPEUTIC USES OF TH1-LIKE REGULATORY T CELLS

(71) Applicant: The Board of Trustees of the Leland Stanford Jun, Palo Alto, CA (US)

(72) Inventors: Dale Umetsu, Newton, MA (US); Rosemarie Dekruyff, Newton, MA (US); Omid Akbari, Los Altos, CA (US); Philippe Stock, Berlin (DE)

(73) Assignee: The Board of Trustees of the Leland Stanford Junior University, Palo Alto, CA (US)

(21) Appl. No.: 13/665,638

(22) Filed: Oct. 31, 2012

Related U.S. Application Data
(63) Continuation of application No. 11/908,419, filed on Mar. 14, 2008, now abandoned.

(51) Int. Cl. C12N 5/0783

(52) U.S. Cl. CPC C12N 5/0637
USPC 424/184.1; 435/375

(57) ABSTRACT

A unique CD4⁺CD25⁺ regulatory T cell population develops from naive CD4⁺CD25⁻ T cells during a T_{H}1 polarized immune response (called T_{H}1-T_{R} cells). These T_{H}1-T_{R} cells can be generated by contacting naïve T cells with mature CD8α⁺ dendritic cells (DCs) that have been exposed to a T_{H}1 polarizing adjuvant and, in some cases, an antigen of interest. The T_{H}1-T_{R} are identified by their expression of the cytokines IL-10 and IFN-γ, the transcriptional regulators T-bet and FoxP3, and the cell surface molecules CD4, CD25, CD69, CD44 and ICOS.

### BACKGROUND OF THE INVENTION

Regulatory CD4⁺ T cells are essential in the control of immune responses. In general, regulatory CD4⁺T cells inhibit the activation and/or function of T helper type 1 (T_{H}1) and T_{H}2 effector cells. Several distinct types of CD4⁺ T regulatory (T_{R}) cells have been described, including CD4⁺CD25⁺ T cells that develop naturally in the thymus which constitute 5-10% of CD4⁺ T cells from naive mice and provide potent inhibitory activity against autoreactive T cells (also called ‘natural T_{R} cells’).

In addition to natural T_{R} cells', several forms of antigen-specific T_{R} cells have been described that are induced after exposure to specific, exogenous antigen (called ‘adaptive T_{R} cells’). These include T_{R} cells that develop in vitro in the presence of interleukin 10 (IL-10; 3) or in the presence of vitamin D3 and dexamethasone, produce IL-10 and inhibit inflammatory responses in the colon and central nervous system. Adaptive T_{R} cells also include antigen-specific T_{R} cells that develop in vivo from CD25⁻ naive T cells after epicutaneous immunization with autoantigenic peptides and inhibit experimental allergic encephalomyelitis or that develop from CD25⁻ naive T cells after respiratory exposure to antigen and inhibit the development of allergen-induced airway hyper-reactivity (AHR). Further, T_{H}3 cells have been described that develop after exposure to oral antigen and inhibit the development of experimental autoimmune encephalomyelitis.

Because adaptive T_{R} cells have been difficult to generate, isolate and study, the relationship between natural and adaptive T_{R} cells; specific methods that efficiently induce the development of adaptive T_{R} cells; and the full range of adaptive T_{R} cells that exist are not fully understood. In light of these deficiencies in our understanding of the nature of T_{R} cells and their biologic activities, the clinical application of T_{R} cells has not been fully realized. As such, it is a goal of the present invention to characterize novel types of adaptive T_{R} cells and provide methods for their generation and therapeutic use.

### RELEVANT LITERATURE

T cells sub-type, including T regulatory cells, are described in the literature, for example by Sakaguchi et al. J. Immunol. 160, 1151-1164 (1995); Bluestone et al. Nat. Rev. Immunol. 3, 253-257 (2003); Groux et al. Nature 389, 737-742 (1997); Barrat et al., J. Exp. Med. 195, 603-616 (2002); Bynoe et al. Immunity 19, 317-328 (2003); Akbari et al. Nat. Med. 8, 1024-1032 (2002); Chen et al. Science 265, 1237-1240 (1994);

Models of airway hyperreactivity are described, for example, by Hansen et al. J. Clin. Invest. 103, 175-183 (1999); Akbari et al. Nat. Immunol. 2, 725-731 (2001); Stock et al. Eur. J. Immunol. 34, 1817-1827 (2004). Methods of inducing a T_{H}1 polarized immune response in a subject are described in U.S. Pat. No. 6,086,898.

The regulatory factor Foxp3 is described by Hori et al. Science 299, 1057-1061 (2003); Fontenot et al. Nat. Immunol. 4, 330-336 (2003). T-bet is described by Szabo et al. Cell 100, 655-669 (2000).

### SUMMARY OF THE INVENTION

Compositions of novel adaptive CD4⁺CD25⁺ regulatory T cell populations and methods for generation and therapeutic use of such T cells are provided. The adaptive regulatory T cells of the invention develop from naive CD4⁺CD25⁻ T cells during a T_{H}1 polarized immune response. These T_{H}1-like regulatory T cells (T_{H}1-T_{R} cells) are generated by contacting naïve T cells with mature CD8α⁺ dendritic cells (DCs) that have been exposed to a T_{H}1 polarizing adjuvant and a specific antigen. The resultant adaptive antigen specific T_{H}1-T_{R} cells are characterized by production of both IL-10 and interferon-γ (IFN-γ), expression of the ‘master T_{H}1 transcription regulator’ T-bet and expression of high levels of inducible costimulator (ICOS). These T_{H}1-T_{R} cells also express Foxp3.

The antigen specific T_{H}1-T_{R} cells of the invention potently inhibit the development of allergen-induced airway hyper-reactivity (AHR). In vitro, these T_{H}1-T_{R} cells block the proliferation and cytokine secretion of both naïve and polarized T cells (e.g., T_{H}1 and T_{H}2 cells). Transplantation of mature CD8α⁺ DCs that have been exposed to a T_{H}1 polarizing adjuvant and an antigen can induce in the recipient the development of antigen specific T_{H}1-T_{R} cells that can inhibit the development of allergen-induced AHR.

Given the ability of these novel T_{H}1-T_{R} cells described herein to inhibit the activation of conventional T cells, they are useful in ameliorating the symptoms of a variety of diseases in which an aberrant immune response is responsible for the disease state, including inflammatory conditions, graft rejection and autoimmunity. Methods are also provided for the induction of T_{H}1-T_{R} by administration of appropriate dendritic cells in vivo. The T_{H}1-T_{R} cells also find use in the analysis of cellular interactions, gene expression, and compound screening relating to modulation of T cell responses.

### BRIEF DESCRIPTION OF THE FIGURES

FIG. 1. CD8α⁺ DCs from mice immunized with OVA+HKL show a mature phenotype. Mice were immunized with OVA or OVA+HKL. After 5 days, CD8α⁺DCs were purified from spleens and analyzed by FACS for expression of cell surface molecules (B7-1, B7-2, MHC class II, ICOS-L, CD40, DEC205, CD8α, B220 and OX40-L). Shaded histogram shows the expression of cell surface molecules in DCs isolated from naïve mice.

FIG. 2. CD11c⁺ CD8α⁺ DCs protect against AHR. (a) CD8α⁻DCs or CD8α⁺DCs, isolated from spleens of BALB/c mice that had been immunized previously with OVA or with OVA plus HKL were adoptively transferred into BALB/c recipients (1×10⁶ cells/mouse), which were then immunized with OVA plus alum. Then 8 d later, mice were challenged intranasally with OVA (50 μg, three times), and AHR was assessed 24 h later. (b,c) The inhibitory function of CD8α⁺DC requires the production of IL-10 and IL-12. CD8α⁺DCs were isolated from IL10^{-/-} (b) or IL12^{-/-} (c) mice (BALB/c background) previously immunized with OVA or with OVA and HKL and were adoptively transferred into wild-type BALB/c mice, as in a. The recipient mice were then immunized with OVA plus alum and 8 d later were challenged intranasally with OVA (50 μg, three times) and were assessed for AHR 24 h later. Results are presented as mean peak Penh values of five mice per group ±s.e.m.

FIG. 3. T cells induced by the regulatory CD8α⁺DC express IL-10 and IFN-γ. (a) CD8α⁺DCs isolated from spleens of BALB/c mice that had been immunized previously with OVA or with OVA plus HKL were adoptively transferred along with naive DO11.10 cells into BALB/c recipient mice. On days 1, 3 and 5, KJ1-26⁺ cells were purified from the spleens of these mice and intracellular cytokine production was assessed by flow cytometry, gating on KJ1-26⁺ cells. Numbers in dot plots represent the percentage of cytokine-producing cells, summarized as graphs (left). Vertical axes on dot plots indicate forward scatter. Results are from one experiment representative of five. (b) As described in a, CD8α⁺DCs from mice previously immunized with OVA plus HKL were adoptively transferred (on day 0) into recipient mice that also received naive DO11.10 cells. Some mice received additional CD8α⁺DCs from mice previously immunized with OVA with or without HKL on days 7 and 14. DO11.10 cells were isolated from the spleens of recipient mice on days 7, 14 and 21, were double-stained for intracellular cytokines and were assessed by flow cytometry, gating on cytokine producing DO11.10 cells. Numbers in quadrants indicate the percentage of cells in that quadrant. Results are from one experiment representative of three.

FIG. 4. TH1-like Regulatory cells secrete IL-10. T_{H}1-like regulatory cells (T_{OVA/HKL} (T_{REG})) were generated with HKL as described in Materials and Methods. T_{H}1-like regulatory cells (1×10⁶ cells/ml) were restimulated with bone marrow-derived DCs (2×10⁴/ml) at the indicated concentrations of OVA in vitro for 96 h. Supernatants were harvested and assessed for IL-10 by ELISA. Activated DO11.10 T_{OVA} cells were generated in the absence of HKL. Naïve DO11.10 cells were used as “negative controls.”

FIG. 5. IL-10-producing T cells express CD25, ICOS, Foxp3 and T-bet. (a) Naive DO11.10 cells (filled histograms) or DO11.10 cells that were adoptively transferred into recipients of CD8α⁺DCs from mice immunized with OVA plus HKL (thick lines) or with OVA (thin lines, as in FIG. 2a) were analyzed for CD25, CD69, CD44, CD62L and ICOS. Filled histograms, naive KJ1-26⁺ T cells (controls). (b) Foxp3 expression in DO11.10 T cells from mice receiving DC_{OVA} (T_{OVA}), CD4⁺CD25⁺ T cells from naive mice (CD4⁺CD25⁺), T_{R} cells producing IFN-γ and IL-10 (DO11.10 T cells from mice receiving DC_{OVA}+HKL; T_{R} listeria), naive DO11.10 cells, T_{R} cells induced by respiratory exposure to OVA (TR pulmonary; 6, 13) and naive CD4+CD25− T cells (CD4+CD25−), determined by real-time RT-PCR and normalized to 18S ribosomal mRNA (reported as comparative fold expression). Data are representative of at least two independent experiments. (c) TR cells producing IFN-γ and IL-10 express T-bet. Left, analysis of T-bet expression in T_{H}1 cells and T_{H}2 cells generated by culture of DO11.10 T cells in T_{H}1-polarizing conditions (thick line) and T_{H}2-polarizing conditions (thin line), respectively. Right, analysis of T-bet expression in control cells and DO11.10 cells from mice receiving DCs stimulated with OVA (T_{OVA}, thin line) and OVA plus HKL (T_{R} listeria, thick line), respectively. Cells were isolated from spleens on day 5 from mice described in FIG. 3b and were treated with phorbol 12-myristate 13-acetate plus ionomycin for 6 h, washed, fixed, permeabilized and stained with either phycoerythrin-isotype antibody (filled histogram) or mAb to T-bet (4B10). (d) GATA3 expression in T_{H}2, T_{H}1, T_{R} listeria, T_{R} pulmonary and T_{OVA} cells, determined by RT-PCR. Results are one experiment representative of three.

FIG. 6. TR cells inhibit AHR and airway inflammation. (a) Mice received either no cells (filled triangles) or DO11.10 T cells plus DCs exposed to OVA plus HKL (open circles) or to OVA alone (filled circles) and were immunized systemically and challenged intranasally with OVA. AHR was assessed 24 h after the last dose of OVA; data are expressed as mean Penh values (±s.e.m.) averaged among five sensitized mice in each group. Results are representative of four independent experiments. (b) T_{R} cells producing IL-10 and IFN-γ inhibit airway inflammation. Lung tissues from recipient mice were sectioned and stained with hematoxylin and eosin (full images) or predigested periodic acid Schiff (insets). All mice were immunized and challenged to OVA, as described. Left, mouse that received TR cells producing IL-10 and IFN-γ (DO11.10 cells from mice receiving DC_{OVA}+HKL) before intranasal challenge with OVA. Middle, mouse that received naive DO11.10 cells before intranasal challenge with OVA. Right, mouse that received control T cells (DO11.10 cells from mice receiving DC_{OVA}) before intranasal challenge with OVA. Original magnification, ×400 (full images) and ×600 (insets). Images are representative sections of five mice per group. (c,d) TR cells were generated and adoptively transferred as described in b. After challenge with OVA, AHR was assessed and results are presented as dynamic compliance of the lung (Cdyn (ml/cm H₂O) (c) and airway resistance (RL (cm H₂O/ml/s) (d). Values were averaged among four mice in each group ±s.e.m. Mice received either IL-10-IFN-γ DO11.10 T_{R} cells (open circles), control DO11.10 T cells generated without HKL (filled circles) or no cells (positive control).

FIG. 7. The regulatory effects of the TR cells depend on IL-10 but not IFN-γ. TR cells were generated and adoptively transferred into mice sensitized to OVA+alum as described in FIG. 6a. Cells were incubated for 4 h at 37° C. with mAb to IL-10 (a) or mAb to IFN-γ (b) and were adoptively transferred together with 500 μg of the same mAb as used for the incubation. AHR was measured after challenge with methacholine; data represent Penh values averaged among sensitized mice in each group.

FIG. 8. TR cells suppress naive and effector T cells. (a) Naive DO11.10 cells (4×10⁴ cells/well) were labeled with CFSE and cultured with bone marrow-derived DCs (1×10⁴) and OVA (250 μg/ml) in the presence of T_{R} cells (1×10⁴) generated with HKL (T_{OVA+HKL}(T_{R})) or control T cells generated without HKL (T_{OVA}). Top, cultures contained no mAb or mAb to IL-10, IFN-γ or ICOSL (100 μg/ml). After 48 h, cells were analyzed by flow cytometry, gated on KJ1-26⁺ cells. Results are one experiment representative of three. (b) T_{H}2 cells (4×10⁴ cells/well) or T_{H}1 cells were cultured with bone marrow-derived DCs (1×10⁴) and OVA (250 μg/ml) and either no other cells (pos. ctrl) or in the presence of T_{R} cells (1×10⁴ cells/well) generated with HKL (T_{OVA+HKL}(T_{R})) or T cells generated without HKL (T_{OVA}) (as in a)+anti-IL-10, addition of neutralizing antibody to IL-10. Supernatants were collected after 96 h and cumulative amounts of cytokines were determined by ELISA. Results are one experiment representative of three.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, preferred methods and materials are described. For purposes of the present invention, the following terms are defined below.

As used herein, by “regulatory T cell” or “T_{R} cell” is meant a T cell of the helper cell lineage (i.e., expressing CD4) that functions to inhibit the activation, growth, and/or the effector function of conventional T cells. T_{R} cells also constitutively express the α chain of the IL-2 receptor (CD25). T_{R} cells have thus far been characterized as either “natural” or “adaptive”, with the “natural” T_{R} cells developing continually in the thymus the “adaptive” T_{R} cells being generated from naïve or memory T cells in the periphery upon exposure to antigen (under certain conditions). The identification and characterization of a novel T_{H}1-type adaptive T_{R} cell is the subject of this application.

As used herein, the term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Preferably, the adjuvant is pharmaceutically acceptable.

Heat killed Listeria adjuvant (HKL), as used herein, is intended to encompass killed Listeria monocytogenes, as well as specific extracts derived therefrom, which are formulated with an immunogen for purposes of immunotherapy. Methods of inactivating Listeria by heat killing, radiation, etc. are known in the art. Listeria extracts, or fractions, that maintain the adjuvant effect of the complete killed bacteria may also be used. Components of interest include Listeria DNA comprising CpG ISS motifs; listeriolysin O, p60, and lipoteichoic acid.

Such extracts have been described in the literature, for example cell wall and peptidoglycan fractions by Paquet et al. (1991; Infection & Immunity 54(1):170-176), various cell wall preparations by Hether et al. (1983; Infection & Immunity 39:1114-1121) and by Schuffler et al. (1976; Immunology 31(2):323-329). Affinity separation methods as known in the art may be used to enrich for adjuvant activity from a complex mixture. The enriched composition may be further purified by preparative gel electrophoresis, HPLC, ion-exchange chromatography, etc.

The dosage of adjuvant may vary depending on the condition of the patient, allergen and specific Listeria compound that is administered. The unit dosage for a single immunization may range from a dose equivalent to from about 10⁵ heat killed Listeria monocytogenes (HKL) per kilogram weight of the recipient, to as much as about 10⁹ equivalents per kilogram weight.

An “immunogenic peptide” or “antigenic peptide” is a peptide which is recognized by a T cell, or which binds an MHC (or other cell surface molecule) to form an epitope recognized by a T cell, thereby inducing a cell mediated response upon presentation to the T cell. Thus, some antigenic peptides are capable of binding to an appropriate MHC molecule and inducing a cytotoxic T cell response, or helper response, e.g., cell lysis or specific cytokine release against the target cell which binds or expresses the antigen, or recruitment of cells to the target cell for subsequent lysis. An “antigenic peptide” can be derived from a polypeptide or protein of varying sizes (or amino acid lengths). The term “antigen” is used to indicate either an antigenic peptide or the polypeptide or protein form which it was derived (or both). In some cases, a polypeptide or protein may contain more than one “antigenic peptide” therein that is presented by MHC molecules and recognized by T cells. For example, a protein associated with tumor cells (i.e., a “tumor antigen”) may have within it 1, 2, 3, 5, 10 or 20 or more “antigenic peptide” sequences that can be presented by an MHC molecule and recognized by a T cell.

A “dendritic cell” (DC) belongs to a group of cells called professional antigen presenting cells (APCs). DCs have a characteristic morphology, with thin sheets (lamellipodia) extending from the dendritic cell body in several directions. Several phenotypic criteria are also typical, but can vary depending on the source of the dendritic cell. These include high levels of MHC molecules (e.g., class I and class II MHC) and costimulatory molecules (e.g., B7-1 and B7-2), and a lack of markers specific for granulocytes, NK cells, B cells, and T cells. Many dendritic cells express certain markers; for example, some Human dendritic cells selectively express CD83, a member of the immunoglobulin superfamily (Zhou and Tedder (1995) Journal of Immunology 3821-3835). Dendritic cells are able to initiate primary T cell responses in vitro and in vivo. These responses are antigen specific. Dendritic cells direct a strong mixed leukocyte reaction (MLR) compared to peripheral blood leukocytes, splenocytes, B cells and monocytes. Dendritic cells are optionally characterized by the pattern of cytokine expression by the cell (Zhou and Tedder (1995) Blood 3295-3301). DCs can be generated in vivo or in vitro from immature precursors (e.g., monocytes).

The terms “high”, “intermediate”, “low”, “positive” or “negative” with respect to the expression of cell surface markers are commonly used in the art to distinguish populations of cells from each other. The subject T_{H}1-T_{R} cells are characterized by their expression of certain cell surface markers. While it is commonplace in the art to refer to cells as “positive” or “negative” for a particular marker, actual expression levels are a quantitative trait. The number of molecules on the cell surface can vary by several logs, yet still be characterized as “positive”. It is also understood by those of skill in the art that a cell which is negative for staining, i.e. the level of binding of a marker specific reagent is not detectably different from a control, e.g. an isotype matched control; may express minor amounts of the marker. Characterization of the level of staining permits subtle distinctions between cell populations.

The staining intensity of cells can be monitored by flow cytometry, where lasers detect the quantitative levels of fluorochrome (which is proportional to the amount of cell surface marker bound by specific reagents, e.g. antibodies). Flow cytometry, or FACS, can also be used to separate cell populations based on the intensity of binding to a specific reagent, as well as other parameters such as cell size and light scatter. Although the absolute level of staining may differ with a particular fluorochrome and reagent preparation, the data can be normalized to a control.

In order to normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining above the brightness of an isotype matched control, but is not as intense as the most brightly staining cells normally found in the population. Low positive cells may have unique properties that differ from the negative and brightly stained positive cells of the sample. An alternative control may utilize a substrate having a defined density of marker on its surface, for example a fabricated bead or cell line, which provides the positive control for intensity.

Therefore, the characterization of cells as expressing specific levels of a particular cell surface antigen (e.g., high, intermediate, or low expression levels) is well known in the art and is used to distinguish cellular populations that possess unique functional characteristics.

Foxp3 is a member of the forkhead/winged-helix family of transcriptional regulators and is highly conserved in humans. The protein product of Foxp3, scurfin, is essential for normal immune homeostasis. The human gene sequence may be accessed at Genbank, AF277993 and is further described by Fontenot et al. (2003) Nat Immunol. 2003 April; 4(4):330-6. Foxp3 is specifically expressed in CD4+CD25+ regulatory T cells and is required for their development. The lethal autoimmune syndrome observed in Foxp3-mutant scurfy mice and Foxp3-null mice results from a CD4+CD25+ regulatory T cell deficiency.

T-bet (T-box 21, Tbx21). Tbx21 is a Th1-specific T-box transcription factor that controls the expression of the hallmark Th1 cytokine, interferon-gamma. TBX21 expression correlates with IFNG expression in Th1 and natural killer (NK) cells. Ectopic expression of TBX21 both transactivated the IFNG gene and induced endogenous IFNG production. The sequence of human TBX21 may be accessed at Genbank, AF241243, and is further characterized by Szabo et al. (2002) Science 295(5553):253. T-bet appears to regulate lineage commitment in CD4 T helper (TH) lymphocytes. T-bet is required for control of IFN-gamma production in CD4 and NK cells, but not in CD8 cells. This difference is also apparent in the function of these cell subsets.

ICOS (activation inducible lymphocyte immunoremediatory molecule) Inducible co-stimulator (ICOS) is a homodimeric protein of relative molecular mass 55,000-60,000 (M(r) 55K-60K). Matching CD28 in potency, ICOS enhances all basic T-cell responses to a foreign antigen, namely proliferation, secretion of lymphokines, upregulation of molecules that mediate cell-cell interaction, and effective help for antibody secretion by B cells. ICOS has to be de novo induced on the T-cell surface, does not upregulate the production of interleukin-2, but superinduces the synthesis of interleukin-10, a B-cell-differentiation factor. In vivo, ICOS is highly expressed on tonsillar T cells, which are closely associated with B cells in the apical light zone of germinal centres, the site of terminal B-cell maturation. See, for example, Hutloff et al. (1999) Nature 397(6716):263-6. The genetic sequence may be accessed at Genbank, AJ277832.

### DETAILED DESCRIPTION OF THE EMBODIMENTS

A unique adaptive CD4⁺CD25⁺ regulatory T cell population that develops from naive CD4⁺CD25⁻ T cells during a T_{H}1 polarized immune response (called T_{H}1-T_{R} cells) is provided herein. The T_{H}1-T_{R} cells of the invention can be generated by contacting naïve T cells with mature CD8α⁺ dendritic cells (DCs) that have been exposed to a T_{H}1 polarizing adjuvant and a specific antigen, by contacting in vitro, or by administration of such dendritic cells in vivo. The T_{H}1-T_{R} cells of the invention can be identified by their expression of the cytokines IL-10 and IFN-γ, the transcriptional regulators T-bet and FoxP3, and the cell surface molecules CD4, CD25, CD69, CD44 and ICOS. In some embodiments, the cells are isolated from a complex population by affinity selection based on one or more of these markers.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. Methods recited herein may be carried out in any order of the recited events that is logically possible, as well as the recited order of events.

All patents and other references cited in this application are incorporated into this application by reference except insofar as they may conflict with those of the present application (in which case the present application prevails). The citation of any publication is for its disclosure prior to the filing date and should not be construed as an admission that the present invention is not entitled to antedate such publication by virtue of prior invention.

### T_{H}1-T_{R} Cells

T_{H}1-T_{R} cells are a subclass of adaptive T_{R} cells (as opposed to natural T_{R} cells) and can be broadly characterized as having the ability to inhibit the activation of conventional T cells, including naïve and memory T cells as well as effector T cells of both the T_{H}1 and T_{H}2 sub-types. TH1-TR cells are so designated because of similarities to conventional T_{H}1 cells as opposed to T_{H}2 cells. For example, T_{H}1-T_{R} cells secrete IFN-γ, a T_{H}1 type cytokine, and not IL-4, a T_{H}2 type cytokine. While the T_{H}1-T_{R} cells of the invention are generated and identified by the methods described herein, T_{H}1-T_{R} cells can be readily identified by virtue of a number of distinct phenotypic and functional characteristics independent of the methods used to generate and/or isolate them. The characteristic features of the T_{H}1-T_{R} cells of the invention are outlined below.

Antigen Specificity.

The T_{H}1-T_{R} cells of the invention are antigen specific, meaning that they express T cell receptors (TCRs) that recognize a specific antigen. In certain embodiments, the antigen is a peptide that is presented in the context of an autologous MHC molecule (e.g., MHC class II). In some of these embodiments, a population of T_{H}1-T_{R} cells that recognize the same antigenic peptide is clonal, meaning that they express identical TCRs (and thus likely derived from a single naïve T cell). In certain other of these embodiments, a population of T_{H}1-T_{R} cells that recognize the same antigenic peptide is polyclonal, meaning that within the population of T_{H}1-T_{R} cells there exists at least two distinct sub-populations that express different TCRs, each of which recognize the same antigen/MHC complex. In some embodiments, a population of T_{H}1-T_{R} cells contains cells that recognize more than one antigenic peptide presented in the context of MHC.

In some embodiments, the antigen recognized by the T_{H}1-T_{R} cells is an allogeneic antigen (e.g., an MHC molecule). As with the antigenic peptide specific T_{H}1-T_{R} cells above, a population of T_{H}1-T_{R} cells that recognizes an allogeneic antigen may be clonal or polyclonal.

Cell surface Markers.

The T_{H}1-T_{R} cells of the invention co-express the helper T cell marker CD4 and the alpha chain of the IL-2 receptor (CD25). In addition to CD4 and CD25 expression, T_{H}1-T_{R} cells express CD69, high levels of the induced costimulatory molecule ICOS (which is associated with IL-10 production in T cells) but low levels of CD62L. T_{H}1-T_{R} cells also express CD44.

Cytokines.

The T_{H}1-T_{R} cells of the invention co-express IL-10, a cytokine associated with T_{R}1 cells, and IFN-γ, which is expressed by conventional helper T cell of the T_{H}1 phenotype.

Transcriptional Regulators.

The T_{H}1-T_{R} cells of the invention express the conventional T_{H}1 cell specific transcriptional regulator T-bet as well as FoxP3, a transcription factor previously associated with “natural” T_{R} cells. In certain embodiments, T_{H}1-T_{R} cells do not express GATA-3, a T_{H}2-specific transcription factor.

Functional Properties.

As mentioned above, the T_{H}1-T_{R} cells of the invention can inhibit the activation of naïve and memory T cells as well as effector T cells of both the T_{H}1 and T_{H}2 sub-types. As such, T_{H}1-T_{R} cells find use in the treatment of a number of diseases states caused by an aberrant immune response, including, but not limited to, allergic reactions, autoimmune conditions, graft versus host disease (GVHD), and rejection of transplanted tissues. Based on our current understanding, the ability of T_{H}1-T_{R} cells to exert their regulatory activity is dependent on their expression (and secretion) of IL-10 and/or expression of ICOS. Specifically, blocking the activity of IL-10 or preventing the engagement of ICOS with its cognate ligand (ICOSL) diminishes the ability of T_{H}1-T_{R} cells to exert their inhibitory function.

The subject T_{H}1-T_{R} cells may be separated from a complex mixture of cells by techniques that enrich for cells having the characteristics as described. T_{H}1-T_{R} cells can also be identified by expression of proteins, for example by immunostaining, functional assay of cytokine production and the like, or by the expression of specific mRNAs by various methods known in the art. Proteins and mRNA corresponding to one or more of the markers described above are of interest for these purposes, e.g. T-bet, Foxp3, ICOS, IL-10, T cell receptor, etc.

For isolation of cells from tissue, an appropriate solution may be used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Separation of the subject cell population may then use affinity separation to provide a substantially pure population. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (propidium iodide, 7-AAD). Any technique may be employed which is not unduly detrimental to the viability of the selected cells.

The affinity reagents may be specific receptors or ligands for the cell surface molecules indicated above. The details of the preparation of antibodies and their suitability for use as specific binding members are well known to those skilled in the art.

Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium which maintains the viability of the cells. A preferred medium is phosphate buffered saline containing from 0.1 to 0.5% BSA. Various media are commercially available and may be used according to the nature of the cells, including Dulbeccos Modified Eagle Medium (dMEM), Hank's Basic Salt Solution (HBSS), Dulbeccos phosphate buffered saline (dPBS), RPMI, Iscoves medium, PBS with 5 mM EDTA, etc., frequently supplemented with fetal calf serum, BSA, HSA, etc.

The labeled cells are then separated as to the phenotype described above. The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscoves medium, etc., frequently supplemented with fetal calf serum.

Compositions highly enriched for T_{H}1-T_{R} activity are achieved in this manner. The subject population will be at or about 50% or more of the cell composition, and usually at or about 90% or more of the cell composition, and may be as much as about 95% or more of the live cell population. The enriched cell population may be used immediately, or may be frozen at liquid nitrogen temperatures and stored for long periods of time, being thawed and capable of being reused. The cells will usually be stored in 10% DMSO, 50% FCS, 40% RPMI 1640 medium. Once thawed, the cells may be expanded by use of growth factors and/or stromal cells for proliferation and differentiation.

The present methods are useful in the development of an in vitro or in vivo model for immune functions and are also useful in experimentation on gene therapy, compound screening, as well as modulation of immune responses.

### T_{H}1-Polarized CD8α⁺DCs

The T_{H}1-polarized CD8α⁺DCs of the present invention are broadly defined as a composition of cells having the capacity to induce T cells, e.g. naïve and/or memory T cells, to develop into T_{H}1-T_{R} cells upon contact in vitro or in vivo. T_{H}1-polarized CD8α⁺DCs express a number of cell surface markers associated with DCs (e.g., CD11c+, MHC class II, CD80, CD86, etc.) as well as CD8α. In addition, T_{H}1-polarized CD8α⁺DCs secrete the cytokines IL-10 and IL-12. In some embodiments, CD8α⁺DCs also secrete TNFα and TGF-β.

In certain embodiments, CD8α⁺DCs present antigen from endogenous antigens while in other embodiments they present exogenous antigen. For example, CD8α⁺DCs can be cultured in vitro in the presence of a specific antigenic peptide for which antigen specific T_{H}1-T_{R} cells are desired. This antigenic peptide is bound by MHC on the surface of the CD8α⁺DCs and is thus in a form to be presented to T cells in such a way as to be recognized by cognate TCR. This is known as “pulsing” DCs with an antigenic peptide and is well known in the art. In addition, DCs can be cultured in the presence of a protein or polypeptide that is internalized, processed, and presented in the context of MHC on the surface of the DC. There are a number of ways known in the art to provide antigen to DCs for the purpose of presentation to T cells, any one of which may be useful in practicing the methods of the present invention.

While virtually any antigen of interest may be used in the methods of the present invention to generate CD8α⁺DCs that induce the development of T_{H}1-T_{R} cells, certain types of antigens are of particular interest. Among these are antigens associated with tumor cells, allergic reactions, autoimmune diseases, transplant rejection (e.g., allogeneic antigens), and infectious agents (e.g., viral or bacterial antigens).

### Methods of Generating T_{H}1-T_{R} Cells

T_{H}1-T_{R} cells of the present invention can be generated both in vivo and in vitro by contacting naïve (or memory) CD4⁺ T cells with T_{H}1-polarized CD8α⁺DCs.

In certain embodiments, T_{H}1-T_{R} cells are generated in vivo in a subject by immunizing the subject with an immunizing composition comprising an antigen and a T_{H}1 polarizing adjuvant. In some of these embodiments, the T_{H}1 polarizing adjuvant is a heat killed Listeria adjuvant (HKL). As demonstrated in the Examples section below, immunization with this composition leads to the development of T_{H}1-polarized CD8α⁺DCs, which can induce the development of T_{H}1-T_{R} cells in the subject that recognize the antigen in the immunizing composition and function to downregulate an immune response to that antigen.

In certain embodiments, T_{H}1-T_{R} cells are generated in vivo by administering T_{H}1-polarized CD8α⁺DCs to a subject. In some of these embodiments, the CD8α⁺DCs are isolated from a donor that has been immunized with a T_{H}1-polarizing immunizing composition. The isolation of the T_{H}1-polarized CD8α⁺DCs from the immunized donor can be achieved using a variety of methods known in the art. In general, a tissue sample containing T_{H}1-polarized CD8α⁺DCs is harvested from the immunized donor (e.g., spleen, lymph node, blood, etc.) and the T_{H}1-polarized CD8α⁺DCs are isolated from other cells in the tissue by virtue of the expression of a specific combination of cell surface markers. In certain of these embodiments, these cell surface markers include CD8α, CD11c, CD80, CD86 and MHC class II.

In other embodiments, T_{H}1-polarized CD8α⁺DCs are generated in vitro. Methods for the generation of DCs in vitro are well known in the art. In general, these methods include isolating/harvesting DC precursors (e.g., monocytes) from a donor (e.g., from peripheral blood) followed by contacting the DC precursors with compositions that promote the development of DCs in culture. To generate T_{H}1-polarized CD8α⁺DCs using these methods, the DC-promoting compositions contain a T_{H}1-polarizing agent (e.g., HKL). In some embodiments, the DC-promoting composition contains the antigen for which specific T_{H}1-T_{R} cells are desired. In certain of these embodiments, the antigen is provided in a form that must be processed by the DC to be presented on the cell surface in MHC molecules (e.g., polypeptide). In other embodiments, the antigen is a peptide that can be directly loaded into cell surface-expressed MHC molecules (e.g., peptide antigen).

Once the desired T_{H}1-polarized CD8α⁺DCs have been generated in vitro, these cells can be administered to a subject in whom development of T_{H}1-T_{R} cells is desired. In certain embodiments, the T_{H}1-polarized CD8α⁺DCs are autologous (or syngeneic) with regard to the subject while in other embodiments they are allogeneic to the subject. In the case of autologous/syngeneic T_{H}1-polarized CD8α⁺DCs, the T_{H}1-T_{R} cells generated in the subject will be specific for antigen(s) presented in the context of MHC. In the case of allogeneic T_{H}1-polarized CD8α⁺DCs, the T_{H}1-T_{R} cells generated in the subject will be specific for the allo-antigen(s) present on the DCs. In certain of these embodiments, the T_{H}1-polarized CD8α⁺DCs are purified prior to being administered to the subject. In some embodiments, the T_{H}1-polarized CD8α⁺DCs are washed and suspended in a physiological medium that promotes the survival of the cells prior to administration to the subject.

The administration of the T_{H}1-polarized CD8α⁺DCs can be achieved using any convenient means that results in the desired outcome: development of T_{H}1-T_{R} cells in the subject. As such, routes of in vivo administration include, but are not limited to, intravenous administration, intraperitoneal administration, intramuscular administration, intranodal administration, intracoronary administration, intraarterial administration (e.g., into a carotid artery), subcutaneous administration, intraventricular administration, intracranial, intraocular, intranasal, and direct injection into a tissue.

In certain embodiments, T_{H}1-T_{R} cells are generated in vitro. The generation of T_{H}1-T_{R} cell in vitro can be achieved in a variety of ways, but in general, these methods involve contacting T_{H}1-polarized CD8α⁺DCs and naïve (or memory) T cells in such a way as to promote the development of T_{H}1-T_{R} cells.

In certain of these embodiments, the T_{H}1-polarized CD8α⁺DCs and the naïve/memory T cells are autologous/syngeneic, meaning that they are derived from the same/genetically identical donor. In other embodiments, the T_{H}1-polarized CD8α⁺DCs are allogeneic with regard to the naïve/memory T cells, meaning that they are derived from different donors.

In certain embodiments, the T_{H}1-polarized CD8α⁺DCs are generated prior to contacting with the naïve/memory T cells. For example, T_{H}1-polarized CD8α⁺DCs can be harvested and isolated from a donor that has been immunized with an immunizing composition comprising an antigen and a T_{H}1-polarizing adjuvant (e.g., HKL). These T_{H}1-polarized CD8α⁺DCs can then be contacted to naïve/memory T cells in vitro such that they develop into T_{H}1-T_{R} cells. As mentioned, the T_{H}1-polarized CD8α⁺DCs may be autologous/syngeneic or allogeneic with regard to the naïve/memory T cells.

In other embodiments, the T_{H}1-polarized CD8α⁺DCs are generated in vitro. In some of these embodiments, the T_{H}1-polarized CD8α⁺DCs are generated prior to contact with the T cells of interest (as described above). In other of these embodiments, the T_{H}1-polarized CD8α⁺DCs develop in the presence of the naïve/memory T cells. For example, dendritic cell precursors and naïve/memory T cells can be harvested and cultured together in vitro in the presence of a T_{H}1-T_{R} cell-promoting composition containing a T_{H}1-polarizing adjuvant (e.g., HKL) such that T_{H}1-T_{R} cells are generated. During the in vitro culture period, T_{H}1-polarized CD8α⁺DCs develop and contact the naïve/memory T cells thereby promoting the development of T_{H}1-T_{R} cells. As mentioned above, the immature DCs and the naïve/memory T cells can be autologous/syngeneic or allogeneic with regard to each other. In some embodiments, the T_{H}1-T_{R} cell-promoting composition contains an antigen for which antigen-specific T_{H}1-T_{R} cells are desired.

### Adoptive Immunotherapy Methods

The methods and compositions of the present invention are useful for inhibiting an aberrant immune response in a subject thereby ameliorating one or a number of disease symptoms. By aberrant immune response is meant the failure of the immune system to distinguish self from non-self or the failure to respond appropriately to foreign antigens. In other words, aberrant immune responses are inappropriately regulated immune responses that lead to disease symptoms in a subject. Diseases or disease conditions that are amenable to treatment using the methods of the subject invention include, but are not limited to, the prevention and treatment of autoimmune diseases, such as inflammatory myopathy, Myasthenia Gravis, inflammatory polyneuropathies, Multiple Sclerosis, asthma, insulin-dependent diabetes mellitus (IDDM), autoimmune thyroiditis, autoimmune gastiritis accompanying pernicious anemia, psoriasis, uveitis, rheumatoid arthritis, Systemic lupus erythematosis (SLE) and colitis. Additional application of this method may be in the prevention of transplant rejection, such as solid organ transplants (kidney, heart, lung, liver, pancreas), cell and tissue transplant rejection (bone marrow transplantation, stem cell transplantation, pancreatic islet transplantation, corneal transplation, lens transplation), graft versus host disease (GVHD) in which transplanted T cells from a donor recognize the recipient as foreign and mount a cytotoxic immune response, and in the treatment of inflammatory diseases, such as inflammatory bowl disorder (IBD), asthma, allergic and atopic reactions.

As used herein, treating a subject using the compositions and methods of the present invention refers to reducing the symptoms of the disease, reducing the occurrence of the disease, and/or reducing the severity of the disease. Treating a subject can refer to the ability of a therapeutic composition of the present invention, when administered to a subject, to prevent a disease from occurring and/or to cure or to alleviate disease symptoms, signs or causes. As such, to treat a subject means both preventing disease occurrence (prophylactic treatment) and treating a subject that has a disease (therapeutic treatment). In particular, treating a subject is accomplished by suppressing an aberrant immune response in the subject.

More specifically, therapeutic compositions as described herein, when administered to a subject by the methods of the present invention, preferably produce a result which can include alleviation of the disease, elimination of the disease, reduction of inflammation associated with the disease, elimination of inflammation associated with the disease, prevention of a secondary disease resulting from the occurrence of a primary disease, and prevention of the disease.

In certain embodiments, in vitro or in vivo generated T_{H}1-polarized CD8α⁺DCs are used as adoptive immunotherapy for amelioration of disease symptoms caused by an aberrant immune response. In these embodiments, T_{H}1-polarized CD8α⁺DCs are administered to a subject to induce the in vivo development of antigen specific T_{H}1-T_{R} cells, thereby ameliorating symptoms caused by an aberrant immune response.

In some of these embodiments, the T_{H}1-polarized CD8α⁺DCs are autologous/syngeneic to the subject and present antigen(s) associated with the aberrant immune response. For example, immature DCs can be harvested from a subject with an aberrant immune response (e.g., an autoimmune disease) and treated in vitro with a T_{H}1-polarizing composition that contains the antigen of interest (e.g., an autoantigen) and a T_{H}1-polarizing adjuvant. The resultant mature T_{H}1-polarized CD8α⁺DCs, which present the antigen of interest, can then be administered to the subject to promote the development of T_{H}1-T_{R} cells in that subject which function to inhibit the aberrant immune response to that antigen. In some embodiments, a single antigen or antigenic peptide is included in the T_{H}1-polarizing composition whereas in other embodiments, more than one antigen or antigenic peptide may be used, including 2, 3, 4, 10 or more. Additionally, multiple independently generated T_{H}1-polarized CD8α⁺DCs can be administered to a subject to inhibit an aberrant immune response in that subject. Furthermore, administration of T_{H}1-polarized CD8α⁺DCs to a subject can be done as often as is required to ameliorate the symptoms associated with the aberrant immune response.

In other of these embodiments, the T_{H}1-polarized CD8α⁺DCs are allogeneic to the subject. For example, immature dendritic cells can be harvested from an organ donor and treated in vitro with a T_{H}1-polarizing composition that contains a T_{H}1-polarizing adjuvant. The resultant allogeneic T_{H}1-polarized CD8α⁺DCs can then be administered to the subject to promote the development of T_{H}1-T_{R} cells in that subject which function to prevent the subjects immune cells from rejecting a transplanted organ derived from the same DC donor.

In certain embodiments, in vivo or in vitro generated T_{H}1-T_{R} cells are used in an adoptive immunotherapy method to ameliorate symptoms associated with an aberrant immune response in a subject.

In certain of these embodiments, the T_{H}1-T_{R} cells are autologous/syngeneic to the subject. For example, naïve and/or memory T cells can be harvested from a subject having an aberrant immune response and cultured in vitro with T_{H}1-polarized CD8α⁺DCs that present the antigen of interest. The antigen specific T_{H}1-T_{R} cells that develop can be purified and administered to the subject where they function to downregulate the aberrant immune response to the antigen of interest.

In other of these embodiments, the T_{H}1-T_{R} cells are allogeneic to the subject being treated for an aberrant immune response. Take for example the case of bone marrow transplantation. In this example, it would be advantageous to produce T_{H}1-T_{R} cells from the donor which can prevent T cells in the transplant material from attacking the recipient leading to the clinical manifestations of GVHD. To do this, CD8α⁺DCs can be isolated/generated from the recipient and contacted with naïve/memory T cells from the donor in in vitro culture under conditions that promote the development of T_{H}1-T_{R} cells. These allogeneic T_{H}1-T_{R} cells can then be purified and administered to the host prior to, in conjunction with, or after administration of the transplant material (e.g., bone marrow cells). The T_{H}1-T_{R} cells would then inhibit activation of the T cells in the transplant material that lead to GVHD.

In certain embodiments of the adoptive immunotherapy methods described above, the cells of interest (i.e., CD8α⁺DCs or T_{H}1-T_{R} cells) can be purified prior to administration to the subject. Purification of the cells can be done using a variety of methods known in the art, including methods in which antibodies to specific cell surface molecules are employed. These methods include both positive and negative selection methods. For example, T_{H}1-T_{R} cells generated in vitro can be isolated by staining the cells with fluorescently labeled antibodies to CD4 and CD25 followed by sorting of the cells that express both of these markers on their cell surface using fluorescence activated cell sorting (FACS). These and other purification/isolation methods are well known to those of skill in the art.

The CD8α⁺DCs or T_{H}1-T_{R} cells of the invention either can be used immediately after their generation (and purification, if applicable) or stored frozen for future use. In certain embodiments, enough CD8α⁺DCs or T_{H}1-T_{R} cells are generated to provide an initial dose for the subject as well as cells that can be frozen and stored for future use if necessary.

In certain other embodiments, CD8α⁺DCs or T_{H}1-T_{R} cells can be expanded in vitro from freshly isolated or frozen cell stocks to generate sufficient numbers of cells for effective adoptive immunotherapy. By effective dose is meant enough cells to ameliorate at least one symptom caused by the aberrant immune response. The determination of an effective dose for therapeutic purposes is known in the art. The expansion of the cells can be achieved by any means that maintains their functional characteristics. For example, a population of antigen-specific T_{H}1-T_{R} cells can be cultured in vitro with T cell mitogens which promote their growth, including agonist antibodies to components of the TCR (e.g., anti-CD3 antibody) and co-stimulatory molecules (e.g., anti-CD28). The phenotypic and functional properties of the resultant expanded cells can be tested prior to their therapeutic use and/or storage to verify that the expansion process has altered their activity.

### Expression Assays

One application of interest is the examination of gene expression in T regulatory cells of the invention. The expressed set of genes may be compared with a variety of cells of interest, e.g. T cells, including TH1 cells, other T_{reg} cells, etc., as known in the art. For example, one could perform experiments to determine the genes that are regulated during development of the regulatory response.

Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A⁺ mRNA. One of skill in the art can readily use these methods to determine differences in the size or amount of mRNA transcripts between two samples. For example, the level of particular mRNAs in Treg cells is compared with the expression of the mRNAs in a reference sample, e.g. T helper cells, or other differentiated cells.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific polynucleotide sequences (or restriction enzyme sites) are used as unique identifiers of genes, coupled with information about fragment length or fragment location within the expressed gene. The relative representation of an expressed gene with in a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with arrays are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

In another screening method, the test sample is assayed at the protein level. Diagnosis can be accomplished using any of a number of methods to determine the absence or presence or altered amounts of a differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods of qualitative or quantitative detection of levels or amounts of differentially expressed polypeptide can be used, for example ELISA, western blot, immunoprecipitation, radioimmunoassay, etc.

### Screening Assays

The subject cells are useful for in vitro assays and screening to detect agents that affect T regulatory cells. A wide variety of assays may be used for this purpose, including toxicology testing, immunoassays for protein binding; determination of cell growth, differentiation and functional activity; production of cytokines; and the like.

In screening assays for biologically active agents the subject cells, usually a culture comprising the subject cells, is contacted with the agent of interest, and the effect of the agent assessed by monitoring output parameters, such as expression of markers, cell viability, and the like. The cells may be freshly isolated, cultured, genetically altered as described above, or the like. The cells may be environmentally induced variants of clonal cultures: e.g. split into independent cultures and grown under distinct conditions, for example with or without the agent; in the presence or absence of other cytokines or combinations thereof. The manner in which cells respond to an agent, particularly a pharmacologic agent, including the timing of responses, is an important reflection of the physiologic state of the cell.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

In addition to complex biological agents, such as viruses, cytokines, antibodies, etc., candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 μl to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluorescence, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

### Examples

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, and pressure is at or near atmospheric.

### Materials and Methods

Mice.

BALB/c and IL-12-deficient mice were purchased from The Jackson Laboratory. IL-10-deficient mice, purchased from The Jackson Laboratory, had a C57BL/6 background and were backcrossed for ten generations to BALB/c in our laboratory. Rag2^{-/-} breeder mice transgenic for an OVA-specific TCR (DO11.10) were provided by A. K. Abbas (Department of Pathology, University of California, San Francisco, San Francisco, Calif.). These mice lack CD25⁺ natural T_{R} cells and were used as donors of OVA-specific CD4⁺CD25⁻ T cells.

Mice were injected intraperitoneally with 200 μg OVA (ICN Biomedical) in incomplete Freund's adjuvant (IFA) or with 200 μg OVA plus 4×10⁸ HKL in IFA on day 0. On day 5, the mice were killed and spleens were collected for further studies. Mice intended to undergo measurement of airway hyperreactivity (AHR) were injected intraperitoneally with OVA (100 μg/mouse) adsorbed to 2 mg of alum. Then, 8 d later, mice were challenged on 3 consecutive days with OVA (three times, each 50 μg/mouse) and AHR was assessed 24 h after the last challenge. The Stanford University Committee on Animal Welfare (Administration Panel of Laboratory Animal Care) approved all animal protocols used in this study.

Isolation, Purification and Adoptive Transfer of Cells.

DCs were isolated by digestion of fragments of spleens at 37° C. for 1 h with a ‘cocktail’ of 0.1% DNase I (fraction IX; Sigma) and 1.6 mg/ml of collagenase (CLS4; Worthington Biochemical) followed by dissociation for 10 min with 10 mM EDTA. CD8α⁺DCs were purified from spleens with the CD8α⁺ Dendritic Cell Isolation Kit (Miltenyi Biotec) according to the manufacturer's instructions. Cells were purified with AutoMACS (Miltenyi Biotec) according to the manufacturer's instructions (purity, >96% by flow cytometry) and cells were injected intravenously into BALB/c recipients (1×10⁶ cells/mouse). Donors of spleen cells were BALB/c, IL-10-deficient or IL-12-deficient mice.

Generation of Regulatory Cells In Vivo.

Mice were injected intraperitoneally with OVA in IFA or with OVA plus HKL in IFA on day 0 (21). On day 5, the mice were killed and spleens were collected for the purification of CD8α⁺DCs stimulated with OVA or OVA plus HKL (CD8α⁺DC_{OVA} or CD8α⁺DC_{OVA+HKL}, respectively). For the generation of regulatory cells, DO11.10 OVA TCR-transgenic CD4⁺CD25⁻ T cells were isolated from spleens of naïve DO11.10 Rag2^{-/-} mice, which do not contain CD25⁺ T_{R} cells, and were injected intravenously into recipients (5×10⁶ cells/mouse). Simultaneously, CD8α⁺DC_{OVA} or CD8α⁺ DC_{OVA+HKL} were injected intravenously into the same recipient mice (1×10⁶ cells/mouse) without further immunization with antigen. Then, 5 d later, DO11.10 cells were isolated by magnetic-activated cell sorting from the spleens of the recipients with mAb KJ1-26 (clonotype specific). In some experiments, those KJ1-26⁺ cells were injected intravenously into mice (3×10⁶ cells/mouse) that had been immunized with OVA and alum (described above) 7 d earlier. These mice were then challenged intranasally with OVA (described above) on three consecutive days, starting 1 d after the transfer of T_{R} cells.

For the depletion of cytokines, T_{R} cells were incubated for 4 h in vitro with 100 mg antibody to IL-10 (anti-IL-10; 2A5), anti-IFN-γ (XMG1.2), anti-ICOSL (16F.7E5) or isotype control antibody. T_{R} cells (3×10⁶) were adoptively transferred intravenously into recipients, which also received 500 μg of the corresponding antibody or isotype control intraperitoneally.

CFSE Labeling and Coculture of Cells.

DO11.10 cells were collected from the spleens of DO11.10 Rag2^{-/-} mice and were labeled with CFSE (Molecular Probes). For assay of regulatory activity, 1×10⁴ regulatory or control cells were cocultured with 4×10⁴ purified and CFSE-labeled DO11.10 cells, T_{H}1 or T_{H}2 cells, in the presence of OVA (250 μg/ml) and 1×10⁴ bone marrow-derived DCs. For some cultures, TR cells were incubated for 4 h in 100 mg of anti-IL-10, anti-IFN-γ, anti-ICOSL or isotype control and were washed before coculture (mAbs were maintained in the cultures at 100 μg/well). After 48 h (CFSE), cells were collected and analyzed by flow cytometry (CFSE). For analysis of cumulative cytokines, cell culture supernatants were collected after 96 h and analyzed by enzyme-linked immunosorbent assay (ELISA). OVA-specific T_{H}1 and T_{H}2 lines were generated from spleens of DO11.10 mice.

Flow Cytometry.

A FACScan (Becton Dickinson) was used for analytical flow cytometry and data were processed with CellQuest Pro (Becton Dickinson) or FlowJo (TreeStar) software. T cells were stained with antibodies to CD44, CD69, CD62L and CD25 (PharMingen) and ICOS and DCs were stained with antibodies to CD80, CD86, CD40, CD8a, B220 (Pharmingen), ICOSL, OX-40L (eBioscience), major histocompatibility complex class II (purified from clone MKD6; American Type Culture Collection) and DEC-205 (Cedarlane Laboratories). Flow cytometry of cytokine production in T cells was done according to a standard protocol, with some modifications. Cells were isolated from spleens and Fc receptors were blocked with excess anti-Fc (HB197). Cell surfaces were stained with fluorescent (fluorescein isothiocyanate or phycoerythrin) or biotin-coupled antibodies, followed by CyChromestreptavidin (PharMingen) where appropriate. Cells were washed twice with cold PBS. For intracellular cytokine assays, T cells were stimulated for 6 h with phorbol 12-myristate 13-acetate (20 ng/ml) plus ionomycin (500 ng/ml). Collected cells were fixed and permeabilized with Cytofix/Cytoperm and Perm/Wash (BD PharMingen) according to the manufacturer's instructions. For staining for cytoplasmic IL-10, IL-4 or IFN-γ (Pharmingen) or for T-bet (57) (4B10; Santa Cruz Biotechnology), the appropriate phycoerythrin-labeled antibodies were added to permeabilized cells (30 min on ice) followed by washing twice with cold PBS.

Cytokine ELISA.

ELISAs were done. The mAb pairs used were as follows, listed as capture—biotinylated detection mAb: IFN-γ, HB170-XMG1.2; IL-4, BVD4-BVD6-24G2; IL-10, SXC.2-SXC.1.

RT-PCR.

For Foxp3 and Gata3 analysis, total RNA was prepared from purified T cells by TRIzol. RT-PCR was done for 30 cycles. The annealing PCR temperature was 57° C. (Foxp3) or 55° C. (Gata3) and the primer sequences were as follows:

Foxp3:
5′-CAGCTGCCTACAGTGCCCCTAG-3′ (forward)
5′-CATTTGCCAGCAGTGGGTAG-3′ (reverse)
Gata3:
5′-AGGCAAGATGAGAAAGAGTGCCTC-3′ (forward)
5′-CTCGACTTACATCCGAACCCGGTA-3′ (reverse)

Real-Time RT-PCR for Foxp3.

For Foxp3 analysis, total RNA was prepared from purified T cells by TrIzol. The DNA was generated. The expression of Foxp3 and 18S ribosomal RNA was quantified by real-time PCR with a sequence detection system (ABI Prism 7900; Applied Biosystems) using the TaqMan 1000 RXN Gold with Buffer A Pack (Applied Biosystems) as well as the following primers and internal fluorescent probes:
Foxp3: 5′-GGCCCTTCTCCAGGACAGA-3′
5′-GCTGATCATGGCTGGGTTGT-3′
5′-5-carboxyfluorescein-ACTTCATGCATCAGCTCTCCACTGTGGAT-N,N,N′,N′-tetramethyl-6-carboxyrhodamine-3′. For both Foxp3 and 18S mRNA quantification, each sample was run in duplicate. Foxp3 mRNA was normalized to 18S mRNA for each sample.

Measurement of Airway Responsiveness.

AHR responses were assessed by methacholine-induced airflow obstruction in conscious mice placed in a whole-body plethysmograph (Buxco Electronics). Peak enhanced pause (Penh) results were confirmed by analysis of AHR in anesthetized and tracheostomized mice, which were mechanically ventilated, with a modified version of published methods. Aerosolized methacholine was administered for 20 breaths in increasing concentrations (1.25, 2.5, 5 and 10 mg/ml of methacholine). Lung resistance and dynamic compliance were continuously computed by fitting of flow, volume and pressure to an equation of motion.

CD8α+ DCs Transfer Suppression

Heat-killed Listeria monocytogenes (HKL) as an adjuvant induces an antigen-specific inhibitory response that prevents the development of and reverses established T_{H}2 responses and AHR. Although HKL induces the development of T_{H}1 cells, the absence of inflammation in the lungs of mice treated with HKL suggests that anti-inflammatory T_{R} cells, rather than proinflammatory T_{H}1 cells, are mainly responsible for the inhibitory effect of HKL on AHR.

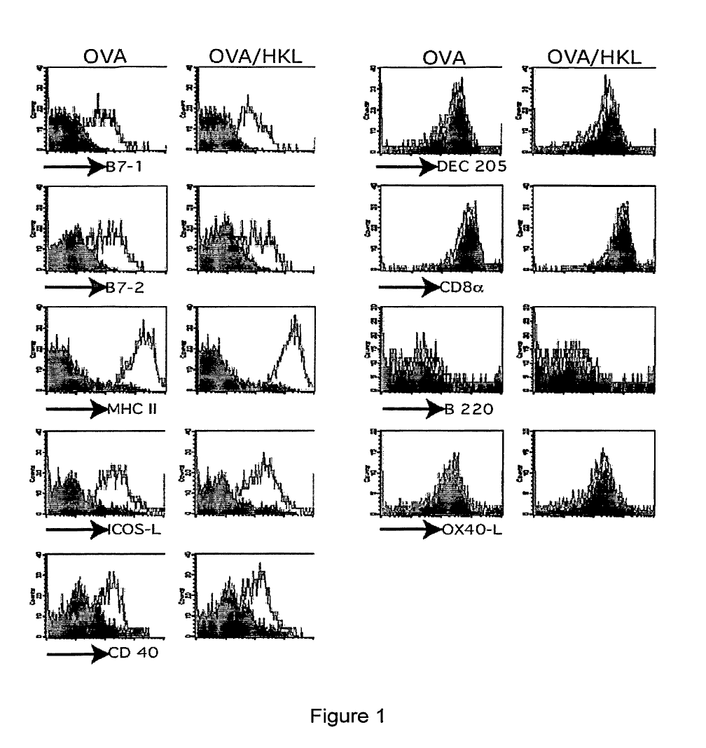

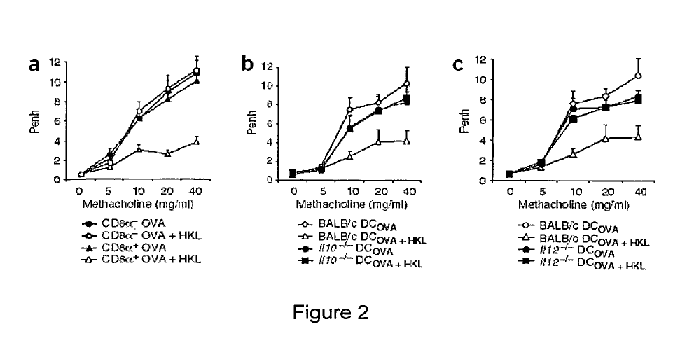

Because the protective effect of HKL is abolished by treatment with mAb to CD8α, we sought to determine if cells expressing CD8α might be responsible for the anti-inflammatory effect of HKL. CD8α⁺DCs from mice immunized with OVA plus HKL had a mature phenotype (high surface expression of CD80, CD86, major histocompatibility complex class II, ICOS ligand (ICOSL), CD40, as well as CD205 (DEC-205) and OX40-L, but not B-220; FIG. 1). Adoptive transfer of these mature CD11c⁺CD8α⁺DCs isolated from mice immunized with OVA plus HKL inhibited the subsequent development of AHR (FIG. 2a), whereas transfer of CD11c⁺CD8α⁻DCs from mice immunized with OVA plus HKL failed to inhibit the development of AHR, indicating that only the CD8α⁺and not the CD8α⁻ DCs were responsible for the inhibitory effect. Furthermore, the inhibitory effect of the CD8⁺ cells was not due to conventional CD8αβ⁺ T cells, as adoptive transfer of CD8⁺ T cells purified with a mAb to CD8β from mice immunized with OVA plus HKL had no inhibitory effect on AHR (data not shown). Thus, CD8α⁺ DCs are effective in transferring the inhibitory effect of HKL, presumably by inducing a regulatory response that inhibited AHR.

We next evaluated the mechanisms by which CD8α⁺ DCs generated by immunization with OVA plus HKL mediated the inhibition of AHR. We examined IL-12 and IL-10 production by the DCs because CD8α⁺ DCs classically produce IL-12, and because regulatory DCs have been shown to produce IL-10 (13). Adoptive transfer of CD8α⁺ DCs isolated from IL-10-deficient mice immunized with OVA plus HKL failed to inhibit AHR (FIG. 2b). Furthermore, adoptive transfer of CD8α⁺ DCs isolated from IL-12-deficient mice immunized with OVA plus HKL also failed to inhibit AHR (FIG. 2c), indicating that the production of both IL-10 and IL-12 by the CD8α⁺ DCs was required for the DCs to exert their protective effects.

Induction of T Cells Producing IL-10 and IFN-γ.

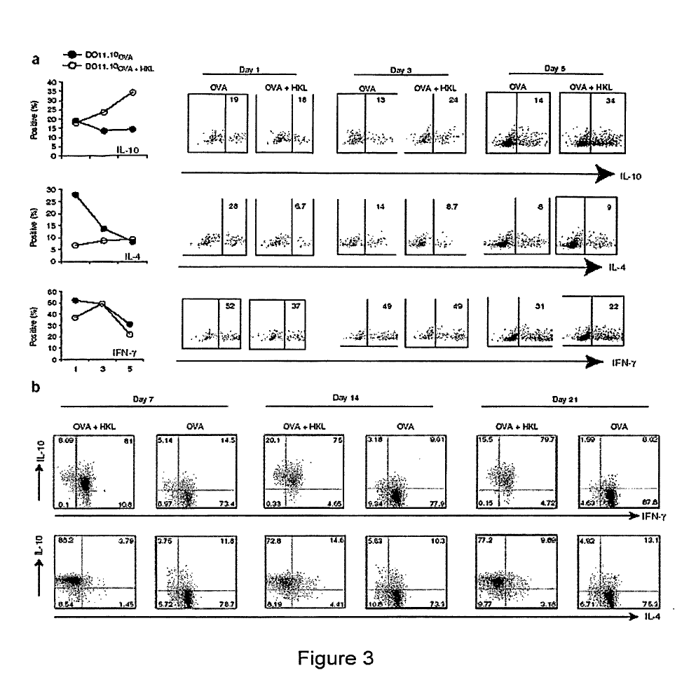

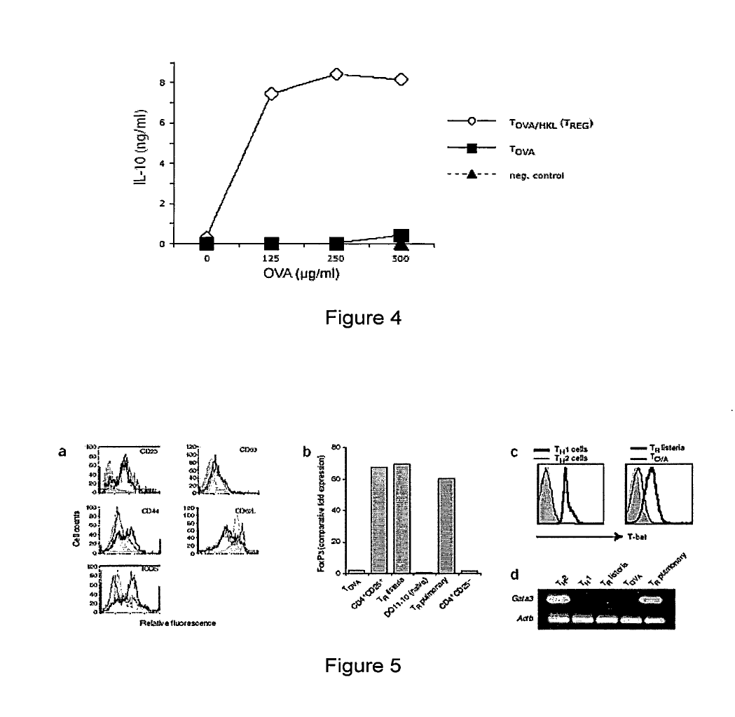

To investigate the mechanism by which CD8α⁺ DCs exert their regulatory effects in this model, we analyzed the T cells activated by the CD8α⁺ DCs. We adoptively transferred CD8α⁺ DCs from mice immunized with OVA plus HKL without further administration of antigen and examined the differentiation in mice that received adoptively transferred naïve OVA-specific CD4+ DO11.10 T cell receptor (TCR)—transgenic T cells (from DO11.10 recombination activating gene 2-deficient (Rag2^{-/-}) mice, which lack CD25⁺ T_{R} cells). Over the course of 5 d, the DO11.10 cells isolated from mice receiving CD8α⁺ DCs exposed to HKL produced large amounts of IL-10 (FIG. 3a and FIG. 4). In contrast, we noted production of IL-4 only in DO11.10 cells at early time points after adoptive transfer of CD8α⁺ DCs exposed to OVA alone. The production of IL-10 in the DO11.10 cells was dependent on the exposure of the DCs to HKL, because the DO11.10 cells that developed in mice receiving CD8α⁺ DC generated in the absence of HKL did not produce IL-10 (FIG. 3a and FIG. 4). In addition, approximately half of the DO11.10 cells generated in the presence of HKL-stimulated DCs produced IFN-γ 3 d after transfer of DCs.

The IFN-γ-producing T cells induced with CD8α⁺ DCs were distinct from T_{H}1 cells, because most of the cytokine-producing DO11.10 examined 7 d after adoptive transfer were positive for both IL-10 and IFN-γ, as shown by intracellular staining of cells positive for KJ1-26, a clonotypic mAb for DO11.10 T cells. These cells producing both IL-10 and IFN-γ did not produce IL-4, as determined by double staining for IL-10 and IL-4. In contrast, the KJ1-26⁺ cells generated in the absence of HKL produced IL-4 and some IFN-γ but not IL-10. The phenotype of T_{R} cells producing both IL-10 and IFN-γ were stable, as production of both cytokines persisted when examined on days 14 and 21, after the mice received additional CD8α⁺ DCs on days 7 and 14 (FIG. 3b).

TR Cells Express ICOS, Foxp3 and T-bet.

To better characterize the T cells producing both IL-10 and IFN-γ, we examined them for expression of other markers of T_{H} cells and T_{R} cells. The T cells producing both IL-10 and IFN-γ expressed CD25, CD44, CD69 and ICOS, a costimulatory molecule associated with IL-10 expression in T cells (6, 15-20), but small amounts of CD62L (FIG. 5a). In contrast, the DO11.10 T cells generated in the absence of HKL expressed CD25, CD69, CD62L, some CD44 and small amounts of ICOS. The T cells producing both IL-10 and IFN-γ generated with HKL-stimulated DCs, but not T cells generated by DCs stimulated only with OVA (T_{OVA}), also expressed mRNA for the transcription factor Foxp3, previously shown to be expressed only by natural CD25⁺ T_{R} cells, as determined by conventional RT-PCR (data not shown) or by quantitative RT-PCR (FIG. 5b). We also noted expression of Foxp3 in another IL-10-producing T_{R} cell type previously demonstrated to develop after respiratory exposure to allergen with CD8α⁻ DCs (T_{R} pulmonary cells; 6). Thus, two different types of antigen-specific adaptive T_{R} cells induced in vivo expressed Foxp3. In contrast, Foxp3 was not expressed by CD25⁻ spleen cells, a CD25⁺ T cell line (IL-2-dependent CTLL) or naive DO11.10 T cells.

We also examined the T cells that were generated with HKL and produced both IL-10 and IFN-γ for expression of the T_{H}1 ‘master transcription regulator’ T-bet and the T_{H}2 ‘master transcription factor’ Gata3. T cells producing both IL-10 and IFN-γ that were generated with HKL, but not those generated in the absence of HKL, expressed T-bet (FIG. 5c). However, these T cells producing both IL-10 and IFN-γ did not express Gata3 (FIG. 5d). In contrast, IL-10-producing T_{R} cells induced by respiratory exposure to allergen (T_{R} pulmonary cells) express GATA3 but not T-bet. Thus, the T cells producing both IL-10 and IFN-γ have characteristics of T_{H}1 cells (expressing T-bet and IFN-γ and generated by CD8α⁺DCs), but are distinct from T_{H}1 cells by having characteristics of T_{R} cells (expressing ICOS, Foxp3 and IL-10). In contrast, the T_{R} pulmonary cells have characteristics of T_{H}2 cells (expressing Gata3 and generated by CD8α⁻ DCs via an IL-4-producing intermediate stage).

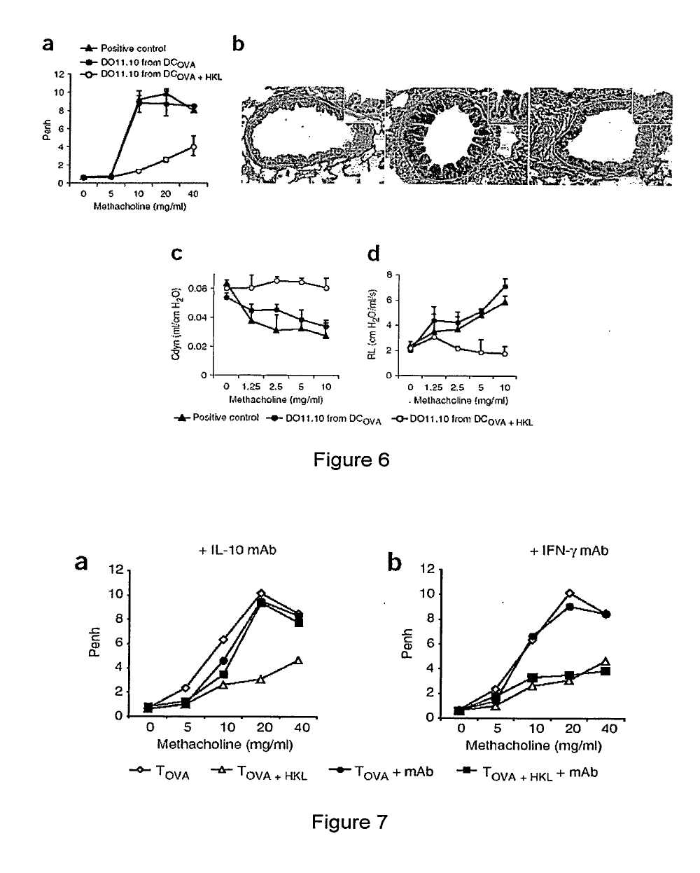

In vivo function of T_{H}1-T_{R} cells. We examined the capacity of the T_{H}1-T_{R} cells producing both IL-10 and IFN-γ to inhibit the development of AHR. We isolated DO11.10 cells from BALB/c mice immunized with CD8α⁺DCs exposed to HKL. We adoptively transferred these OVA-specific KJ1-26⁺ cells into recipients that had been sensitized with OVA in Al(OH)₃ (alum) 8 d before. At 24 h after transfer, we challenged the recipient mice intranasally with OVA to induce AHR. Adoptive transfer of T_{H}1-T_{R} cells notably reduced the development of AHR, whereas transfer of control T cells generated with DCs in the absence of HKL did not (FIG. 6a). The reduction in AHR by the T_{H}1-T_{R} cells was accompanied by a notable reduction in airway inflammation (FIG. 6b). Thus, transfer of the T_{H}1-T_{R} cells but not naive DO11.10 cells greatly reduced the peribronchiolar infiltrate and mucus production in the airways (FIG. 6b). Transfer of T cells generated with DCs in the absence of HKL also did not inhibit airway inflammation, such that large numbers of inflammatory cells and abundant mucus in pulmonary epithelial cells were present in the airways (FIG. 6b). We confirmed the inhibitory effect of the TH1-T_{R} cells on AHR by assessing AHR using direct invasive assays for dynamic compliance (FIG. 6c) and lung resistance (FIG. 6d) in mice that were anesthetized, tracheostomized and mechanically ventilated.

The anti-inflammatory effects of the transferred cells were not due to conventional antigen-specific T_{H}1 cells, because adoptively transferred T_{H}1 cells cannot inhibit, but instead greatly exacerbate, airway inflammation and AHR in sensitized mice. Moreover, the inhibitory effects of the T_{H}1-T_{R} cells were blocked by a mAb to IL-10 (FIG. 7a) but not by a mAb to IFN-γ (FIG. 7b), indicating that the production of IL-10 and not IFN-γ by the T_{H}1-T_{R} cells was required for their regulatory effects. We confirmed the requirement for IL-10 but not IFN-γ production by the T_{H}1-T_{R} cells to reduce AHR by invasive measurements of compliance and resistance of the lungs). Thus, T_{H}1-T_{R} cells are distinct from T_{H}1 cells and have a potent anti-inflammatory function that reverses established T_{H}2 responses.

Analysis of the In Vitro Function of T_{H}1-T_{R} Cells.

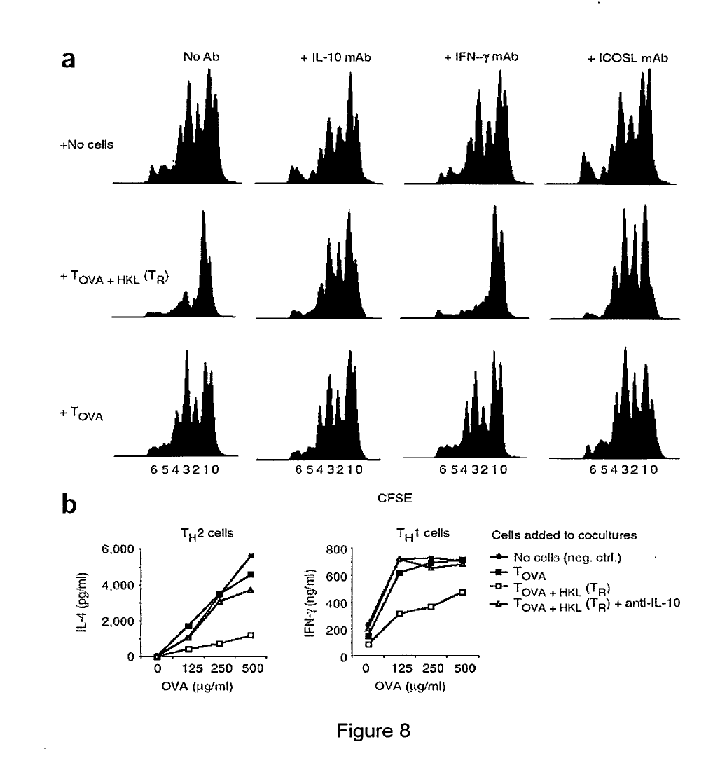

To further analyze the suppressive capacity of T_{H}1-T_{R} cells, we examined their effects on naive DO11.10 T cells and on OVA-specific T_{H}1 and T_{H}2 effector cells. In the absence of the T_{H}1-T_{R} cells, naive DO11.10 cells labeled with 5- (and 6-) carboxyfluorescein diacetate succinimidyl diester (CFSE) proliferated vigorously in response to DCs plus OVA, completing three to four rounds of cell division over 48 h (FIG. 8a). The addition of the T_{H}1-T_{R} cells notably inhibited the proliferation of the CFSE-labeled cells. The inhibitory effect of T_{H}1-T_{R} cells was dependent on IL-10 and the ICOS-ICOSL pathway, because the addition of neutralizing mAb to IL-10 or mAb to ICOSL to the cultures restored the proliferation of naive DO11.10 T cells. In contrast, the addition of mAb to IFN-γ produced little or no effect on the function of T_{H}1-T_{R} cells. Control T cells generated in the absence of HKL (T_{OVA} cells) did not inhibit the proliferation of the naive DO11.10 T cells. Thus, the T_{H}1-T_{R} cells inhibit antigen-specific T cell proliferation in an IL-10- and ICOS-dependent but IFN-γ-independent way.

To investigate whether T_{H}1-T_{R} cells could inhibit the function of polarized effector T cells, we cultured established OVA-specific T_{H}1 or T_{H}2 cells in the presence or absence of T_{H}1-T_{R} cells. The addition of T_{H}1-T_{R} cells reduced the production of IL-4 by T_{H}2 cells and IFN-γ by T_{H}1 cells (FIG. 8b). In contrast, the addition of control T cells generated in the absence of HKL to the cultures did not alter the release of IL-4 by T_{H}2 cells or of IFN-γ by T_{H}1 cells. The inhibitory effects of the T_{H}1-T_{R} cells on effector T_{H}2 and T_{H}1 cells were dependent on IL-10, because neutralization of IL-10 inhibited their suppressive effects and restored the production of IL-4 and IFN-γ, respectively. These results demonstrate that T_{H}1-T_{R} cells have functions distinct from those of T_{H}1 cells, in that they inhibit the proliferation of naive cells and suppress IL-4 and IFN-γ production in polarized effector T cells in vitro in an ICOS- and IL-10-dependent way.

### Discussion

The results described herein identify a unique antigen-specific adaptive T_{R} cell producing both IL-10 and IFN-γ that was induced by mature CD8α⁺DCs. The cytokine profile of these T_{R} cells, their expression of T-bet and the requirement for CD8α⁺DCs suggest that these T_{R} cells are related to T_{H}1 cells. However, these T_{H}1-T_{R} cells are distinct from T_{H}1 cells because they potently inhibited established T_{H}2 responses and allergen-induced AHR, a function that cannot be accomplished by conventional T_{H}1 cells. Moreover, these T_{H}1-T_{R} cells expressed IL-10 and ICOS, which were required for their function, and Foxp3, a transcription factor that was previously thought to be restricted to CD25⁺ T_{R} cells but that we find is common to T cells with potent regulatory capacities.

The adjuvant used in our studies to induce T_{H}1-T_{R} cells, HKL, potently induces T_{H}1 responses that might counter allergic responses mediated by T_{H}2 cells. However, the potency of HKL as an adjuvant to inhibit established T_{H}2-driven inflammatory responses is not solely due to the development of T_{H}1 responses but also is due to the development of a T_{H}1-T_{R} cell response. The inhibitory effect of HKL on AHR and airway inflammation was blocked not only by neutralization of IL-12 but also by neutralization of IL-10, demonstrating that T_{R} cells are involved. In addition, although mAb to CD8α abolishes the inhibitory effect of HKL on AHR and airway inflammation, we have shown here that the CD8⁺ cells that mediated the HKL effect are CD8α⁺ DCs producing IL-10 as well as IL-12 and not CD8⁺ T cells, which have the capacity in some systems to protect against airway hyperreactivity. Also, conventional T_{H}1 cells by themselves are ineffective in dampening established T_{H}2 responses, because T_{H}1 cells in the respiratory mucosa are proinflammatory rather than anti-inflammatory. Instead, T cells producing IL-10 or transforming growth factor-β have much greater anti-inflammatory activity and are much more effective in limiting airway inflammation and hyperreactivity. Therefore, HKL is a complex adjuvant that potently induces not only conventional T_{H}1 responses but also modified T_{H}1 responses characterized by T_{R} cells producing IFN-γ and IL-10.

The combined production of IL-10 and IFN-γ in the T_{H}1-T_{R} cells can be a synergistic combination that inhibits effector T cell responses. Production of IFN-γ in combination with IL-10 has been shown to be induced in T cells by IL-12 and by certain intracellular pathogens such as leishmania, borrelia or mycobacteria. The combined production of IL-10 with IFN-γ occurs in immunoregulatory T cells that protect against severe inflammatory pathology and that help to maintain pathogen-specific immunological memory. We have demonstrated that involvement of CD8α⁺DCs producing both IL-10 and IL-12 is essential in the induction of such T_{R} cells.

The expression of different costimulatory molecules on distinct types of DCs greatly influences the type of T_{R} cell that develops during an immune response. For example, immature CD8α⁻ or CD8α⁺ DCs expressing limited quantities of costimulatory molecules have been linked to the induction of tolerance and to the silencing of pathogenic self-reactive CD4⁺ or CD8⁺ T cells that have escaped negative selection in the thymus, by inducing anergy or deletion, or the development of regulatory cells. In contrast, plasmacytoid (B220⁺) DCs, characterized by their potential to secrete large amounts of type I interferons in response to viral infection, as well as mature CD8α⁻ DCs in the respiratory tract, maintain tolerance by inducing adaptive T_{R} cells. In addition, DC exposed to Bordetella pertussis produce IL-10 and induce bordetella-specific T_{R} cells. Our studies have demonstrated that mature CD8α⁺ DCs producing IL-12, which had been thought to induce mainly T_{H}1 cell differentiation rather than tolerance, can in fact induce T_{H}1-T_{R} cells.

The T_{H}1-T_{R} cells described herein have similarities to both conventional T_{H}1 cells and to previously described T_{R} cells that developed in the respiratory tract from naive CD4⁺CD25⁻ T cells after respiratory exposure to antigen. Both respiratory-induced T_{R} cells (T_{R} pulmonary) and T_{H}1-T_{R} cells are derived from naïve CD4⁺CD25⁻ T cells, express the transcription factor Foxp3 and potently inhibit the development of AHR by pathways involving IL-10 and the regulatory ICOS-ICOSL signaling pathway. However, these two adaptive T_{R} cell types are distinct because the respiratory induced T_{R} cells are induced with CD8α⁻ DCs, they express GATA3 and they developed through a stage in which they transiently produced IL-4, making them a T_{H}2 lineage regulatory cell. The T_{H}1-T_{R} cells of the present invention are induced with CD8α⁺ DCs, do not express GATA3 and do not developed through a stage in which they transiently produced IL-4. Thus, it is clear that there exists a spectrum of antigen-specific adaptive T_{R} cells, which develop in under distinct immune response conditions, and include T_{R} cells related to both the T_{H}1 (T_{H}1-T_{R} cells) and T_{H}2 (T_{H}2-T_{R} cells) lineage of helper T cells.

Expression of both IL-10 and Foxp3 is a characteristic that has been most closely related to CD25⁺ natural T_{R} cells. However, the expression of FoxP3 has not been shown to be a defining characteristic of adaptive T_{R} cells. For example, T_{R} cells induced with myelin basic protein peptide were shown not to produce either IL-10 or Foxp3. Further, IL-10-secreting T_{R} cells induced with IL-10 do not express Foxp3. In contrast, prolonged subcutaneous infusion of a low dose of peptide with an osmotic pump implanted in mice transforms mature T cells into CD4⁺CD25⁺ T_{R} cells that do express Foxp3.

In summary, we have described a previously unknown adaptive T_{R} cell type that expresses IFN-γ, T-bet, IL-10 and Foxp3 and has a potent inhibitory function. These T_{R} cells develop under T_{H}1-biased conditions from naïve and/or memory T cells when stimulated with T_{H}1 polarized CD8α⁺ DCs. These T_{H}1-T_{R} cells inhibit the activation of a wide variety of T cell responses (e.g., both the T_{H}1 and T_{H}2 T cell responses) and as such find use in ameliorating the symptoms of disease states in which an aberrant immune response is the cause.

What is claimed is:

1. A composition of regulatory T cells (T_{H}1-T_{R} cells), wherein said T_{H}1-T_{R} cells:
constitutively express the cell surface markers CD4 and CD25;
secrete the cytokines IL-10 and IFN-γ;
recognize a specific antigen; and
are capable, upon antigenic stimulation, of inhibiting the activation, growth, and/or effector function of conventional T cells.

2. The T_{H}1-T_{R} cells according to claim 1, wherein said T_{H}1-T_{R} cells are further characterized as expressing T-bet.

3. The T_{H}1-T_{R} cells according to claim 1, wherein said T_{H}1-T_{R} cells are further characterized as expressing FoxP3.

4. The T_{H}1-T_{R} cells according to claim 1, wherein said T_{H}1-T_{R} cells are further characterized as not expressing GATA3.

5. The T_{H}1-T_{R} cells according to claim 1, wherein said T_{H}1-T_{R} cells are further characterized as expressing high levels of ICOS.

6. The T_{H}1-T_{R} cells according to claim 1, wherein said T_{H}1-T_{R} cells are further characterized as expressing CD69.

7. The T_{H}1-T_{R} cells according to claim 1, wherein said T_{H}1-T_{R} cells are further characterized as expressing CD44.

8. The T_{H}1-T_{R} cells according to claim 1, wherein said T_{H}1-T_{R} cells are further characterized as expressing low levels of CD62L.

9. A method of producing T_{H}1-T_{R} cells as described in claim 1, said method comprising:
generating T_{H}1-polarized CD8α⁺DCs presenting an antigen; and
contacting said T_{H}1-polarized CD8α⁺DCs to naïve and/or memory T cells;
wherein T_{H}1-T_{R} cells specific for said antigen are produced from said naïve and/or memory T cells.

10. The method according to claim 9, wherein said contacting said T_{H}1-polarized CD8α⁺DCs to said naïve and/or memory T cells occurs in vivo.

11. The method according to claim 9, wherein said contacting said T_{H}1-polarized CD8α⁺DCs to said naïve and/or memory T cells occurs in vitro.

12. The method according to claim 9, wherein said antigen is a peptide antigen presented in the context of an autologous MHC molecule.

13. The method according to claim 9, wherein said antigen is an allo-antigen.

14. The method according to claim 9, wherein said generating step comprises contacting precursors of mature dendritic cells to an immunizing composition comprising a T_{H}1-polarizing adjuvant, wherein said T_{H}1-polarized CD8α⁺DCs presenting said antigen develop from said precursors of mature dendritic cells.

15. The method according to claim 14, wherein said contacting said precursors of mature dendritic cells to said immunizing composition occurs in vitro.

16. The method according to claim 14, wherein said contacting said precursors of mature dendritic cells to said immunizing composition occurs in vivo.

17. The method according to claim 14, wherein said T_{H}1-polarizing adjuvant comprises heat killed Listeria.

18. The method according to claim 14, wherein said immunizing composition further comprises said antigen.

19. A method of inhibiting an aberrant immune response to an antigen in a subject, said method comprising:
generating T_{H}1-polarized CD8α⁺DCs presenting said antigen; and
administering said T_{H}1-polarized CD8α⁺DCs to said subject;
wherein T_{H}1-T_{R} cells specific for said antigen develop in said subject and inhibit said aberrant immune response.

20. The method according to claim 19, wherein said generating step comprises contacting precursors of mature dendritic cells to an immunizing composition comprising a T_{H}1-polarizing adjuvant, wherein said T_{H}1-polarized CD8α^{α}DCs presenting said antigen develop from said precursors of mature dendritic cells.

21. The method according to claim 19, wherein said T_{H}1-polarized CD8α⁺DCs are purified prior to said administration to said subject.

22. The method according to claim 20, wherein said contacting said precursors of mature dendritic cells to said immunizing composition occurs in vitro.

23. The method according to claim 20, wherein said contacting said precursors of mature dendritic cells to said immunizing composition occurs in vivo.

24. The method according to claim 20, wherein said T_{H}1-polarizing adjuvant comprises heat killed Listeria.

25. The method according to claim 19, wherein said antigen is a peptide antigen presented in the context of an autologous MHC molecule.

26. The method according to claim 19, wherein said antigen is an allo-antigen.

27. The method according to claim 20, wherein said immunizing composition further comprises said antigen.

28. The method according to claim 18, wherein said aberrant immune response is chosen from the group consisting of autoimmune diseases, allergic reactions, inflammatory responses, graft versus host disease and transplant rejection.

29. A method of inhibiting an aberrant immune response to an antigen in a subject comprising administering to said subject T_{H}1-T_{R} cells specific for said antigen as described in claim 1 wherein said aberrant immune response to said antigen is inhibited in said subject.

30. The method according to claim 29, wherein said T_{H}1-T_{R} cells are produced as described in claim 9.

31. The method according to claim 29, wherein said antigen is a peptide antigen presented in the context of an autologous MHC molecule.

32. The method according to claim 29, wherein said antigen is an allo-antigen.

33. The method according to claim 29, wherein said aberrant immune response is chosen from the group consisting of autoimmune diseases, allergic reactions, inflammatory responses, graft versus host disease and transplant rejection.

34. A composition of T_{H}1-polarized CD8α⁺DCs, wherein said T_{H}1-polarized CD8α⁺DCs:
have a mature dendritic cell phenotype;
secrete the cytokines IL-10 and IL-12;
are derived from precursors of mature dendritic cells in the presence of a T_{H}1-polarizing adjuvant; and
promote the development of antigen specific T_{H}1-T_{R} cells from naïve and/or memory T cells when contacted to said naive and/and or memory T cells in vitro or in vivo.


SEQUENCE LISTING (7 sequences)

SEQ ID NO: 1 - DNA, 22 bp, Artificial sequence
  Synthesized Primer
cagctgcctacagtgcccctag

SEQ ID NO: 2 - DNA, 20 bp, Artificial sequence
  Synthesized Primer
catttgccagcagtgggtag

SEQ ID NO: 3 - DNA, 24 bp, Artificial sequence
  Synthesized Primer
aggcaagatgagaaagagtgcctc

SEQ ID NO: 4 - DNA, 24 bp, Artificial sequence
  Synthesized Primer
ctcgacttacatccgaacccggta

SEQ ID NO: 5 - DNA, 19 bp, Artificial sequence
  Fluorescent probe
ggcccttctccaggacaga

SEQ ID NO: 6 - DNA, 20 bp, Artificial sequence
  Fluorescent probe
gctgatcatggctgggttgt

SEQ ID NO: 7 - DNA, 29 bp, Artificial sequence
  Fluorescent probe
acttcatgcatcagctctccactgtggat